(19) United States
(12) Patent Application Publication
(10) Pub. No.: US 2015/0134266 A1
(43) Pub. Date: May 14, 2015

(54) METHOD OF IDENTIFYING THE MOLECULAR BASIS OF THE ACTIVITY OF A DRUG COMPOUND, PHARMACEUTICAL COMPOSITIONS, AND TREATMENT METHODS

(71) Applicants: Timothy J. CARDOZO, New York, NY (US); Evgeny V. SHMELKOV, New York, NY (US)

(72) Inventors: Timothy J. CARDOZO, New York, NY (US); Evgeny V. SHMELKOV, New York, NY (US)

(73) Assignee: NEW YORK UNIVERSITY, New York, NY (US)

(21) Appl. No.: 14/538,950

(22) Filed: Nov. 12, 2014

Related U.S. Application Data
(60) Provisional application No. 61/903,071, filed on Nov. 12, 2013.

(51) Int. Cl. G06F 19/00; A61K 31/343; G06F 19/18; A61K 31/165

(52) U.S. Cl. CPC G06F 19/702; A61K 31/165; A61K 31/343; G06F 19/701; G06F 19/18
USPC 702/19; 514/468

(57) ABSTRACT

The present invention relates to a method of identifying the molecular basis of a drug compound's activity. The invention further relates to a pharmaceutical composition, a kit for treating psychosis in a subject, and a method for treating psychosis and/or schizophrenia.

This application claims the benefit of U.S. Provisional Patent Application Ser. No. 61/903,071, filed Nov. 12, 2013, which is hereby incorporated by reference in its entirety.

This invention was made with government support under NIH Grant No. 1RC2LM010994-01. The government has certain rights in this invention.

### FIELD OF THE INVENTION

The present invention relates to a method of identifying the molecular basis of a drug compound's activity, pharmaceutical compositions, and treatment methods.

### BACKGROUND OF THE INVENTION

Enormous quantities of expression, sequence, and proteomic data characterizing both normal and diseased tissues are accumulating, leading to the development of more and more complex molecular biomarkers for diseases. However, the majority of drugs in current clinical use were discovered by phenotypic screens, resulting in their precise mechanism of action being unknown. A knowledge gap exists between the efforts to characterize disease and the chemicals used to treat them (FIG. 1).

Polypharmacology has been gaining increasing attention in the field of drug discovery (Peters, J. U. Polypharmacology in Drug Discovery (2011)). It was reported in the literature that at least some approved drugs exhibit polypharmacological signatures by interacting with multiple targets (Keiser et al., “Relating Protein Pharmacology by Ligand Chemistry,” Nat. Biotechnol. 25:197-206 (2007); Keiser et al., “Predicting New Molecular Targets for Known Drugs,” Nature 462:175-181 (2009); Ashburn et al., “Drug Repositioning: Identifying and Developing New Uses for Existing Drugs,” Nat. Rev. Drug Discov. 3:673-683 (2004); Durrant et al., “A Multidimensional Strategy to Detect Polypharmacological Targets in the Absence of Structural and Sequence Homology,” PLoS Comput. Biol. 6:e1000648 (2010); Mestres et al., “The Topology of Drug-target Interaction Networks: Implicit Dependence on Drug Properties and Target Families,” Mol. Biosyst. 5:1051-1057 (2009); Yang et al., “Chemical-protein Interactome and Its Application in Off-target Identification,” Interdiscip. Sci. 3:22-30 (2011)). The identification of multiple protein targets is essential both for understanding the full mechanism of action of drugs and for side-effect (toxicity) prediction. Moreover, the development of compounds that interact with multiple targets is appealing in case of complex multi-genetic diseases, such as cancer (Knight et al., “Targeting the Cancer Kinome Through Polypharmacology,” Nat. Rev. Cancer 10:130-137 (2010)) and psychiatric disorders (Allen et al., “Strategies to Discover Unexpected Targets for Drugs Active at G Protein-coupled Receptors,” Annu. Rev. Pharmacol. Toxicol. 51:117-144 (2011); Brown et al., “Systems Biology and Systems Chemistry: New Directions for Drug Discovery,” Chem. Biol. 19:23-28 (2012); Metz et al., “Rational Approaches to Targeted Polypharmacology: Creating and Navigating Protein-ligand Interaction Networks,” Curr. Opin. Chem. Biol. 14:498-504 (2010)). The full polypharmacological profile of a drug can be identified only by comprehensive analysis of drug-target interactions on a proteome-wide scale (Xie et al., “Novel Computational Approaches to Polypharmacology as a Means to Define Responses to Individual Drugs,” Annu. Rev. Pharmacol. Toxicol. 52:361-379 (2012)). Matching complex molecular (multi-target) biomarkers of disease to drugs may require complete polypharmacologic fingerprints for the drugs.

In recent years, large databases of compound-protein bioactivities have become available (Wang et al., “PubChem: A Public Information System for Analyzing Bioactivities of Small Molecules,” Nucleic Acids Res. 37:W623-633 (2009); Seiler et al., “ChemBank: A Small-molecule Screening and Cheminformatics Resource Database,” Nucleic Acids Res, 36:D351-359 (2008); Ihlenfeldt et al., “Enhanced CACTVS Browser of the Open NCI Database,” J. Chem. Inf. Comput. Sci. 42:46-57 (2002); Knox et al., “DrugBank 3.0: A Comprehensive Resource for ‘Omics’ Research on Drugs,” Nucleic Acids Res. 39:D1035-1041 (2011); Kanehisa et al., “KEGG for Representation and Analysis of Molecular Networks Involving Diseases and Drugs,” Nucleic Acids Res. 38:D355-360 (2010); Zhu et al., “Therapeutic Target Database Update 2012: A Resource for Facilitating Target-oriented Drug Discovery,” Nucleic Acids Res. 40:D1128-1136 (2012); Gaulton et al., “ChEMBL: A Large-scale Bioactivity Database for Drug Discovery,” Nucleic Acids Res, 40:D1100-1107 (2012); Liu et al., “BindingDB: A Web-accessible Database of Experimentally Determined Protein-ligand Binding Affinities,” Nucleic Acids Res. 35:D198-201 (2007); Hecker et al., “SuperTarget Goes Quantitative: Update on Drug-target Interactions,” Nucleic Acids Res. 40:D1113-1117 (2012); Wang et al., “The PDBbind Database: Methodologies and Updates,” J. Med. Chem. 48:4111-4119 (2005); Benson et al., “Binding MOAD, a High-quality Protein-ligand Database,” Nucleic Acids Res. 36:D674-678 (2008); Block et al., “AffinDB: A Freely Accessible Database of Affinities for Protein-ligand Complexes from the PDB,” Nucleic Acids Res. 34:D522-526 (2006); Kuhn et al., “STITCH 2: An Interaction Network Database for Small Molecules and Proteins,” Nucleic Acids Res 38:D552-556 (2010); Scheer et al., “BRENDA, the Enzyme Information System in 2011,” Nucleic Acids Res 39:D670-676 (2011); Roth et al., “Magic Shotguns Versus Magic Bullets: Selectively Non-selective Drugs for Mood Disorders and Schizophrenia,” Nat. Rev. Drug Discov. 3:353-359 (2004); Sharman et al., “IUPHAR-DB: New Receptors and Tools for Easy Searching and Visualization of Pharmacological Data,” Nucleic Acids Res. 39:D534-538 (2011); Garcia-Serna et al., “iPHACE: Integrative Navigation in Pharmacological Space,” Bioinformatics 26:985-986 (2010); Okuno et al., “GLIDA: GPCR—Ligand Database for Chemical Genomics Drug Discovery—Database and Tools Update,” Nucleic Acids Res. 36:D907-912 (2008)). These databases can be divided into two major classes: (i) all databases that contain data primarily generated by high-throughput screening (HTS) experiments (Wang et al., “PubChem: A Public Information System for Analyzing Bioactivities of Small Molecules,” Nucleic Acids Res. 37:W623-633 (2009); Seiler et al., “ChemBank: A Small-molecule Screening and Cheminformatics Resource Database,” Nucleic Acids Res. 36:D351-359 (2008); Ihlenfeldt et al., “Enhanced CACTVS Browser of the Open NCI Database,” J. Chem. Inf. Comput. Sci. 42:46-57 (2002); Roth et al., “Magic Shotguns Versus Magic Bullets: Selectively Non-selective Drugs for Mood Disorders and Schizophrenia,” Nat. Rev. Drug Discov. 3:353-359 (2004); Garcia-Serna et al., “iPHACE: Integrative Navigation in Pharmacological Space,” Bioinformatics 26:985-986 (2010); Okuno et al., “GLIDA: GPCR—Ligand Database for Chemical Genomics Drug Discovery—Database and Tools Update,” Nucleic Acids Res. 36:D907-912 (2008)), and (ii) repositories that are specifically focused on extracting experimental binding data from literature (Knox et al., “DrugBank 3.0: A Comprehensive Resource for ‘Omics’ Research on Drugs,” Nucleic Acids Res. 39:D1035-1041 (2011); Kanehisa et al., “KEGG for Representation and Analysis of Molecular Networks Involving Diseases and Drugs,” Nucleic Acids Res. 38:D355-360 (2010); Zhu et al., “Therapeutic Target Database Update 2012: A Resource for Facilitating Target-oriented Drug Discovery,” Nucleic Acids Res. 40:D1128-1136 (2012); Gaulton et al., “ChEMBL: A Large-scale Bioactivity Database for Drug Discovery,” Nucleic Acids Res. 40:D1100-1107 (2012); Liu et al., “BindingDB: A Web-accessible Database of Experimentally Determined Protein-ligand Binding Affinities,” Nucleic Acids Res. 35:D198-201 (2007); Hecker et al., “SuperTarget Goes Quantitative: Update on Drug-target Interactions,” Nucleic Acids Res. 40:D1113-1117 (2012); Wang et al., “The PDBbind Database: Methodologies and Updates,” J. Med. Chem. 48:4111-4119 (2005); Benson et al., “Binding MOAD, a High-quality Protein-ligand Database,” Nucleic Acids Res. 36:D674-678 (2008); Block et al., “AffinDB: A Freely Accessible Database of Affinities for Protein-ligand Complexes from the PDB,” Nucleic Acids Res. 34:D522-526 (2006); Kuhn et al., “STITCH 2: An Interaction Network Database for Small Molecules and Proteins,” Nucleic Acids Res. 38:D552-556 (2010); Scheer et al., “BRENDA, the Enzyme Information System in 2011,” Nucleic Acids Res. 39:D670-676 (2011); Sharman et al., “IUPHAR-DB: New Receptors and Tools for Easy Searching and Visualization of Pharmacological Data,” Nucleic Acids Res. 39:D534-538 (2011); Garcia-Serna et al., “iPHACE: Integrative Navigation in Pharmacological Space,” Bioinformatics 26:985-986 (2010); Okuno et al., “GLIDA: GPCR—Ligand Database for Chemical Genomics Drug Discovery—Database and Tools Update,” Nucleic Acids Res. 36:D907-912 (2008)). For the purpose of deriving holistic polypharmacologic fingerprints, the subset of this data pertaining to human protein interactions is relevant. However, in spite of the availability of this large set of data, the complete interaction network between drugs/drug-like compounds and human proteins (receptors/targets) and, thus, the complete polypharmacological profiles of marketed drugs and drug candidates has not been assembled.

Even if the universe of compound-human protein bioactivity scores were available, multiple informatics elements would be needed to identify the physiologically significant receptors/targets of a drug or compound from among all of the recorded bioactivity scores. Normalization schemes, appropriate data structures, and statistical models would need to be implemented in order to generalize comparisons to all compounds and all receptors/targets, as well as to identify statistically significant interactions as a proxy for physiologically significant interactions. Recently, a number of ligand-based and structure-based in silico approaches emerged to address the off-target identification aspect of this issue (Keiser et al., “Predicting new Molecular Targets for Known Drugs,” Nature 462:175-181 (2009); Durrant et al., “A Multidimensional Strategy to Detect Polypharmacological Targets in the Absence of Structural and Sequence Homology,” PLoS Comput. Biol. 6:e1000648 (2010); Gao et al., “PDTD: A Web-accessible Protein Database for Drug Target Identification,” BMC Bioinformatics 9:104 (2008); Abdulhameed et al., “Exploring Polypharmacology Using a ROCS-Based Target Fishing Approach,” J. Chem. Inf. Model (2012); Nettles et al., “Bridging Chemical and Biological Space: ‘Target Fishing’ Using 2D and 3D Molecular Descriptors,” J Med. Chem. 49:6802-6810 (2006); Kinnings et al., “ReverseScreen3D: A Structure-based Ligand Matching Method to Identify Protein Targets,” J. Chem. Inf. Model 51:624-634 (2011); Mestres et al., “Ligand-based Approach to In Silico Pharmacology: Nuclear Receptor Profiling,” J. Chem. Inf. Model 46: 2725-2736 (2006); Cleves et al., “Robust Ligand-based Modeling of the Biological Targets of Known Drugs,” J. Med. Chem. 49:2921-2938 (2006); Rognan, “Chemogenomic Approaches to Rational Drug Design,” Br. J. Pharmacol. 152:38-52 (2007); Rognan, “Structure-Based Approaches to Target Fishing and Ligand Profiling,” Mol. Inform. 29:176-187 (2010); Li et al., “BioDrugScreen: A Computational Drug Design Resource for Ranking Molecules Docked to the Human Proteome,” Nucleic Acids Res. 38:D765-773 (2010); Li et al., “Docking Small Molecules to Predicted Off-Targets of the Cancer Drug Erlotinib Leads to Inhibitors of Lung Cancer Cell Proliferation with Suitable In Vitro Pharmacokinetic Properties,” ACS Med. Chem. Lett. 1:229-233 (2010); Chen et al., “Ligand-protein Inverse Docking and its Potential Use in the Computer Search of Protein Targets of a Small Molecule,” Proteins 43:217-226 (2001)). Ligand-based approaches are based on an assumption that chemically similar structures are more likely to have similar pharmacological profiles. Several successful stories of ligand-based off-target identification exist (Keiser et al., “Predicting New Molecular Targets for Known Drugs,” Nature 462:175-181 (2009); Abdulhameed et al., “Exploring Polypharmacology Using a ROCS-Based Target Fishing Approach,” J. Chem. Inf. Model (2012); Nettles et al., “Bridging Chemical and Biological Space: ‘Target Fishing’ Using 2D and 3D Molecular Descriptors,” J. Med. Chem. 49:6802-6810 (2006); Kinnings et al., “ReverseScreen3D: A Structure-based Ligand Matching Method to Identify Protein Targets,” J. Chem. Inf. Model 51:624-634 (2011); Mestres et al., “Ligand-based Approach to In Silico Pharmacology: Nuclear Receptor Profiling,” J. Chem. Inf. Model 46: 2725-2736 (2006); Cleves et al., “Robust Ligand-based Modeling of the Biological Targets of Known Drugs,” J. Med. Chem. 49:2921-2938 (2006); Rognan, “Chemogenomic Approaches to Rational Drug Design,” Br. J. Pharmacol. 152:38-52 (2007)). Moreover, a number of web databases that allow the user to search by chemical similarity and/or substructure are available (Keiser et al., “Relating Protein Pharmacology by Ligand Chemistry,” Nat. Biotechnol. 25:197-206 (2007); Wang et al., “PubChem: A Public Information System for Analyzing Bioactivities of Small Molecules,” Nucleic Acids Res. 37:W623-633 (2009); Seiler et al., “ChemBank: A Small-molecule Screening and Cheminformatics Resource Database,” Nucleic Acids Res. 36:D351-359 (2008); Ihlenfeldt et al., “Enhanced CACTVS Browser of the Open NCI Database,” J. Chem. Inf. Comput. Sci. 42:46-57 (2002); Knox et al., “DrugBank 3.0: A Comprehensive Resource for ‘Omics’ Research on Drugs,” Nucleic Acids Res. 39:D1035-1041 (2011); Zhu et al., “Therapeutic Target Database Update 2012: A Resource for Facilitating Target-oriented Drug Discovery,” Nucleic Acids Res. 40:D1128-1136 (2012); Gaulton et al., “ChEMBL: A Large-scale Bioactivity Database for Drug Discovery,” Nucleic Acids Res. 40:D1100-1107 (2012); Liu et al., “BindingDB: A Web-accessible Database of Experimentally Determined Protein-ligand Binding Affinities,” Nucleic Acids Res. 35:D198-201 (2007); Wang et al., “The PDBbind Database: Methodologies and Updates,” J. Med. Chem. 48:4111-4119 (2005); Kuhn et al., “STITCH 2: An Interaction Network Database for Small Molecules and Proteins,” Nucleic Acids Res. 38:D552-556 (2010); Kinnings et al., “ReverseScreen3D: A Structure-based Ligand Matching Method to Identify Protein Targets,” J. Chem. Inf. Model 51:624-634 (2011)).

One idea related to the structure-based off-target identification approaches is inverse docking (Chen et al., “Ligand-protein Inverse Docking and its Potential Use in the Computer Search of Protein Targets of a Small Molecule,” Proteins 43:217-226 (2001)), where a single compound is docked to multiple targets and the potential biological targets are ranked based on the docking (Durrant et al., “A Multidimensional Strategy to Detect Polypharmacological Targets in the Absence of Structural and Sequence Homology,” PLoS Comput. Biol. 6:e1000648 (2010); Gao et al., “PDTD: A Web-accessible Protein Database for Drug Target Identification,” BMC Bioinformatics 9:104 (2008); Li et al., “BioDrugScreen: A Computational Drug Design Resource for Ranking Molecules Docked to the Human Proteome,” Nucleic Acids Res. 38:D765-773 (2010); Li et al., “Docking Small Molecules to Predicted Off-Targets of the Cancer Drug Erlotinib Leads to Inhibitors of Lung Cancer Cell Proliferation with Suitable In Vitro Pharmacokinetic Properties,” ACS Med. Chem. Lett. 1:229-233 (2010); Chen et al., “Ligand-protein Inverse Docking and its Potential Use in the Computer Search of Protein Targets of a Small Molecule,” Proteins 43:217-226 (2001); Grinter et al., “An Inverse Docking Approach for Identifying New Potential Anti-cancer Targets,” J. Mol. Graph Model 29:795-799 (2011); Paul et al., “Recovering the True Targets of Specific Ligands by Virtual Screening of the Protein Data Bank,” Proteins 54:671-680 (2004)) or Z-score (Yang et al., “SePreSA: A Server for the Prediction of Populations Susceptible to Serious Adverse Drug Reactions Implementing the Methodology of a Chemical-protein Interactome,” Nucleic Acids Res. 37:W406-412 (2009)). Various success stories of structure-based off-target identification via inverse docking have been reported (Rognan, “Structure-Based Approaches to Target Fishing and Ligand Profiling,” Mol. Inform. 29:176-187 (2010); Li et al., “Docking Small Molecules to Predicted Off-Targets of the Cancer Drug Erlotinib Leads to Inhibitors of Lung Cancer Cell Proliferation with Suitable In Vitro Pharmacokinetic Properties,” ACS Med. Chem. Lett. 1:229-233 (2010); Chen et al., “Ligand-protein Inverse Docking and its Potential Use in the Computer Search of Protein Targets of a Small Molecule,” Proteins 43:217-226 (2001); Grinter et al., “An Inverse Docking Approach for Identifying New Potential Anti-cancer Targets,” J. Mol. Graph Model 29:795-799 (2011); Paul et al., “Recovering the True Targets of Specific Ligands by Virtual Screening of the Protein Data Bank,” Proteins 54:671-680 (2004)).

The growing databases of experimental bioactivity scores improves the feasibility of using this method to identify all possible targets for all known drugs and drug-like compounds by computational approaches. However, a gap would still remain between the polypharmacology of a drug and its pharmacodynamics: the distribution of its receptor targets in the human body. Specifically, in order for the affinity of a drug for a receptor to be significant in a disease, the receptor must be expressed in a tissue relevant to the disease. Thus, for example, expression of a target in bone is not relevant to the action of a schizophrenia drug, no matter how high the affinity of the drug for the receptor. The true fingerprint of drug action (drug pharmacodynamics) is the ensemble of receptors for which a drug has affinity that are expressed in the tissues of the human body relevant to the disease, which is the holistic fingerprint for drug action.

The present invention is directed to overcoming the above-noted and other deficiencies in the art.

### SUMMARY OF THE INVENTION

A first aspect of the present invention relates to a method of characterizing the molecular basis of a drug compound's activity. This method involves selecting a drug compound. A molecular target computing device is used to identify molecular targets with which the drug compound interacts. Identifying involves obtaining from a database comprising compound-target bioactivities a first and second molecular target for the drug. The expression levels of the first and second molecular targets are compared to identify the molecular basis of the drug compound's activity. The molecular target computing device may operate based on existing data or by generating new data by docking.

A second aspect of the present invention relates to a pharmaceutical composition comprising a melatonergic agonist, a “typical” anti-psychotic drug, and a pharmaceutically acceptable carrier.

A third aspect of the present invention relates to a kit for treating psychosis in a subject that includes a melatonergic agonist, a “typical” anti-psychotic drug, and instructions for treating psychosis in the subject by administering the melatonergic agonist and the “typical” anti-psychotic drug.

A fourth aspect of the present invention relates to a method of treating a subject for schizophrenia. This method involves administering to the subject a melatonergic agonist and a “typical” anti-psychotic to treat the subject for schizophrenia.

### BRIEF DESCRIPTION OF THE DRAWINGS

FIG. 1 is a schematic illustration showing the knowledge gap that exists in the prior art between the efforts to characterize diseases and the drug compounds used to treat them.

FIG. 2 is a schematic illustration of one embodiment of a molecular target computing device according to a method of the present invention.

FIG. 3 is a schematic illustration showing one embodiment of one component of a method of the present invention for characterizing the molecular basis of a drug compound's activity. In particular, “PharmaReceptOmic” scores, defined as a set of scores describing the full polypharmacologic action of a drug, are derived from compound-target bioactivity data.

FIG. 4A is a schematic illustration showing one embodiment of a second component of a method of the present invention for characterizing the molecular basis of a drug compound's activity. In particular, the integration of target-ligand interaction probability scores with gene expression data to produce HistoReceptOmics scores is shown. The thickness of the arrows represents the strength of affinity between a drug compound and molecular targets. The left box displays gene expression data of molecular targets. The darker the shading the higher the level of expression. The right box displays combined tissue-molecular scores calculated based on the mathematical equation presented above the arrow (S=−log₁₀Kᵢ×Z), where Kᵢ is affinity and Z is a normalized gene expression level. FIG. 4B shows the statistical model of HistoReceptOmic profile of a drug compound, such that certain scores can be identified as statistically significant. The distribution of statistically significant combined tissue-molecular scores reveals the HistoReceptOmic profile of a drug compound.

FIG. 5 is a schematic illustration showing the analysis of the entire dataset of potential human receptors of clozapine and chlorpromazine affinity (Ki) data for 34 proteins targeted by clozapine and 41 proteins targeted by chlorpromazine. This affinity data was combined with gene expression data for each protein target in 77 normal human tissues to obtain tissue-target signatures for both drugs.

FIG. 6 shows the target signature for the anti-psychotic effect of clozapine and chlorpromazine obtained from the analysis described in FIG. 5.

### DETAILED DESCRIPTION OF THE INVENTION

The present invention is directed to methods for resolving the molecular activity of a drug compound by using a molecular target computing device to identify molecular targets from a compound-target bioactivity database, and then comparing the identified molecular target(s) to expression data for the molecular target(s) across human tissues. The present invention is further directed to pharmaceutical compositions, treatment kits, and treatment methods relating to drug compounds whose molecular activity was resolved using the above method of the present invention.

A first aspect of the present invention relates to a method of characterizing the molecular basis of a drug compound's activity. This method involves selecting a drug compound. A molecular target computing device is used to identify molecular targets with which the drug compound interacts. Identifying involves obtaining from a database comprising compound-target bioactivities a first and second molecular target for the drug compound. The expression levels of the first and second molecular targets are compared to identify the molecular basis of the drug compound's activity.

In carrying out this method of the present invention, “characterizing the molecular basis of a drug compound's activity” means developing a “fingerprint” of the drug compound's action in a biological system. For example, according to one embodiment, characterizing the molecular basis of a drug compound's activity involves identifying the ensemble of molecular targets (e.g., receptors) for which the drug compound has affinity and determining expression (e.g., rate, location, timing, etc.) of the molecular targets in the biological system.

Selecting a drug compound in carrying out the method of the present invention may involve, for example, selecting a compound with a known activity, a known structure, known targets, or any combination thereof. Selecting a compound may, on the other hand, involve choosing a compound with only one of a known structure or known activity.

A “drug compound” according to the present invention is any compound known to be useful or potentially useful (but not yet known to be useful) for the treatment or prevention of a disease or disorder or as a component or potential component of a medication. Alternatively, a “drug compound” is any compound that affects the function of a biological system. Drug compounds thus include, without limitation: small molecules of research or therapeutic interest; naturally-occurring factors, such as endocrine, paracrine, or autocrine factors or factors interacting with cell receptors of all types; intracellular factors, such as elements of intracellular signaling pathways; factors isolated from other natural sources; and so forth.

As used herein, a “molecular target” refers to any cell component whose function is modified by interaction with a drug. Molecular targets may include, but are not limited to, proteins, nucleic acids, lipids or other intracellular or extracellular components. In a one embodiment, the molecular target is a protein. In another embodiment, the molecular target is a protein selected from a membrane protein and a soluble protein.

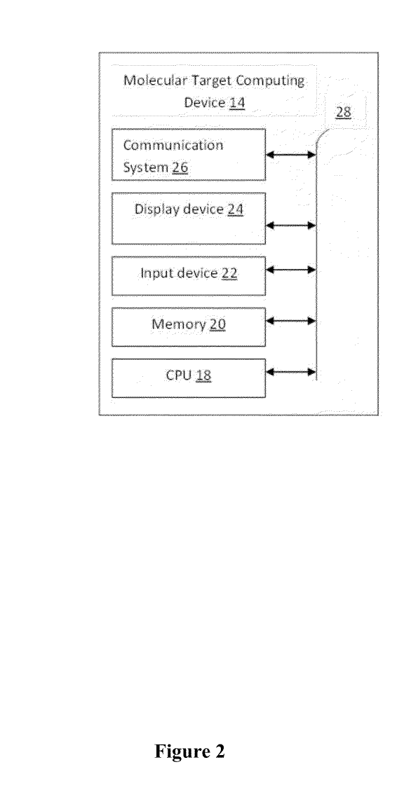

This method of the present invention involves using a molecular target computing device to identify molecular targets with which the drug compound interacts. As illustrated in the particular embodiment shown in FIG. 2, the molecular target computing device 14 includes central processing unit (CPU) or processor 18, memory 20, input device 22, display device 24, and a communication system 26, which are coupled together by bus 28 or other link, although other numbers and types of systems, devices, components, and elements in other configurations and locations can be used. In this particular embodiment, bus 28 is a hyper-transport bus, although other bus types and links may be used, such as PCI.

Processor 18 in molecular target computing device 14 executes a program of stored instructions for one or more aspects of carrying out this method of the present invention as described and illustrated by way of the examples herein, although other types and numbers of processing devices and logic could be used and processor 18 could execute other numbers and types of programmed instructions.

Memory 20 in molecular target computing device 14 stores these programmed instructions for one or more aspects of the method of the present invention as described and illustrated herein, although some or all of the programmed instructions could be stored and executed elsewhere. A variety of different types of memory storage devices, such as a random access memory (RAM) or a read-only memory (ROM) in the system or a floppy disk, hard disk, CD ROM, DVD ROM, or other computer readable medium which is read from and written to by a magnetic, optical, or other reading and writing system that is coupled to the processor in the molecular target computing device 14 could be used.

Input device 22 enables a user, such as a researcher, to interact with molecular target computing device 14, such as to input data and/or to configure, program, and/or operate molecular target computing device 14. By way of example only, input device 22 may include one or more of a touch screen, keyboard, and/or a computer mouse.

Display device 24 enables a user, such as researcher, to interact with molecular target computing device 14, such as to view and/or input data and/or to configure, program, and/or operate molecular target computing device 14 by way of example only. For example, the display device 24 may include one or more of a touch screen display, a CRT, an LED monitor, or an LCD monitor, although other types and numbers of display devices could be used.

Communication system 26 in molecular target computing device 14 is used to operatively couple and communicate between molecular target computing device 14, databases described infra, servers, and/or other devices, such as, smartphones, handheld devices, computing devices, etc., which are all coupled together via the communications network (not shown). Other types and numbers of systems, devices, or elements and other types and numbers of communication networks or systems with other types and numbers of connections and configurations can be used. In one embodiment, communication system 26 is a Wide Area Network (WAN), or a Local Area Network (LAN), although the communication system can include other types of topologies supporting communication. By way of example only, communications system 26 can use TCP/IP over Ethernet and industry-standard protocols, including NFS, CIFS, SOAP, XML, LDAP, and SNMP, although other types and numbers of communication networks, such as a direct connection, a local area network, a wide area network, modems and phone lines, e-mail, and wireless communication technology, each having their own communications protocols, can be used.

Two or more molecular target computing devices can be used in carrying out this method of the present invention. Principles and advantages of distributed processing, such as redundancy and replication also can be implemented, as desired, to increase the robustness and performance of the molecular target computing device. The molecular target computing device may be part of a larger computer system or systems that extend across any suitable network using any suitable interface mechanisms and communications technologies, including for example, telecommunications in any suitable form (e.g., voice and modem), wireless communications media, wireless communications networks, cellular communications networks, G3 communications networks, Public Switched Telephone Network (PSTNs), Packet Data Networks (PDNs), the Internet, intranets, and combinations thereof.

A non-transitory computer readable medium having instructions stored thereon may be used for one or more aspects of this method of the present invention, which, when executed by a processor, causes the processor to carry out the steps necessary to implement the steps for carrying out the inventive method.

According to one embodiment, the molecular target computing device may be used to carry out other aspects of the method of the present invention including, without limitation, selecting the drug compound and comparing molecular targets to expression levels of the molecular targets.

According to one embodiment, expression levels of the molecular target(s) may be in normal, non-disease tissue. According to another embodiment, expression levels of the molecular targets are in disease tissue. According to yet another embodiment, expression levels of the molecular target(s) are in both non-disease and disease tissue or tissue that is not known to be diseased or non-diseased.

In carrying out this method of the present invention, the drug compound may have a known or unknown molecular target. In one embodiment, new molecular targets are discovered in carrying out this method of the present invention, or no new molecular targets are discovered.

In the method of the present invention, the molecular target computing device is used to identify molecular targets with which the drug compound interacts. This involves obtaining from a database one or more molecular targets. For example, the computing device obtains (e.g., provides information to a user regarding) molecular targets from a library of compound-target bioactivities. These data may be stored on a single or separate databases. The data in the database(s) may be part of the computing device (e.g., stored in its memory) or merely accessible by the computing device and stored at one or more remote locations and accessible via, e.g., internet communication.

In one embodiment, compound-target bioactivities are associated with data ranking the relative strength and/or weakness of a molecular target's interaction with the drug compound to that of another molecular target's interaction with the drug compound. These data exist, for example, in libraries identified in the examples discussed infra. An algorithm is developed that selects bioactivities at a particular threshold of relative strength to identify only the most relevant molecular targets.

According to one embodiment, comparing the affinity of a compound for first and second molecular targets to expression levels of the first and second molecular targets is carried out using an algorithm. Specifically, the expression levels of any given molecular target from their genes across human tissues is normally distributed for non-significant tissues/expressions. By comparing the expression level of a given molecular target in a specific tissue to this normal distribution, statistically significant increases in expression of the molecular target in those tissues can be identified. These statistically significant increases identify the particular tissue as likely being biologically responsive to the molecular target or to drugs binding to and modulating the molecular target. The extent of deviation from the normal distribution for any one expression level can be measured by the Z-score, which is a commonly used statistical measure equal to the difference between the expression value and the mean of the normal distribution divided by the standard deviation of the normal distribution. Thus, a molecular target expressed in a single tissue, e.g. pancreas, at a level three (3) standard deviations above the mean of the expression levels of all other tissues has a Z-score of 3.0. In the algorithm, to compare the affinity of a compound for first and second molecular targets to the expression levels of those first and second molecular targets from their genes, the Z-score is simply multiplied by the affinity or a logarithmic transformation of the affinity.

This method of the present invention is carried out, according to one embodiment, to identify a tissue in which the first and second molecular targets exist.

In another embodiment, the method of the present invention further involves comparing the molecular basis of a first drug compound to the molecular basis of a second drug compound. The second drug compound may have side effects not associated with the first drug compound. For example, the first drug compound may have side effects the second drug compound does not have.

In one embodiment, the drug compound is used to treat psychosis. As used herein, the term “psychosis” refers to a psychiatric symptom, condition, or syndrome in its broadest sense, as defined in the DSM-IV-TR comprising a “psychotic” component. The term psychosis can refer to a symptom associated with a general medical condition, a disease state, or other condition, such as a side effect of drug abuse (a substance-induced disorder) or as a side effect of a medication. Alternatively, the term psychosis can refer to a condition or syndrome not associated with any disease state, medical condition, drug intake, or the like.

Psychosis is typically defined as a mental disorder or condition causing gross distortion or disorganization of a person's mental capacity, affective response, or capacity to recognize reality, communicate, and relate to others to the degree of interfering with the capacity to cope with the ordinary demands of everyday life.

Another aspect of the present invention is directed to a pharmaceutical composition comprising a melatonergic agonist, a “typical” anti-psychotic drug, and a pharmaceutically acceptable carrier.

According to this aspect of the present invention, the melatonergic agonist may be any melatonergic agonist known in the art or yet to be discovered. In one embodiment, the melatonergic agonist is selected from the group consisting of agomelatine and ramelteon.

“Typical” anti-psychotics used for the purposes of the present invention may include, but are not limited to, haloperidol, chlorpromazine, chlorprothixene, fluphenazine, loxapine, mesoridazine, perphenazine, pimozide, thioridazine, thiothixene, trifluoperazine, and trifluopromazine. As used herein, a “typical” anti-psychotic is a drug compound that exhibits a reliable anti-psychotic action accompanied by consistent extrapyramidal and endocrine side effects that have been ascribed to their high affinity dopamine D2 receptor antagonism.

The pharmaceutical composition of the present invention further contains, in addition to a melatonergic agonist and a “typical” anti-psychotic drug, a pharmaceutically acceptable carrier and, optionally, other pharmaceutically acceptable components (see REMINGTON'S PHARMACEUTICAL SCIENCE (19th ed., 1995), which is hereby incorporated by reference in its entirety). The incorporation of such pharmaceutically acceptable components depends on the intended mode of administration and therapeutic application of the pharmaceutical composition. Typically, however, the pharmaceutical composition will include a pharmaceutically-acceptable, non-toxic carrier or diluent, which is defined as a vehicle commonly used to formulate pharmaceutical compositions for animal or human administration. The diluent is selected so as not to affect the biological activity of the composition. Exemplary carriers or diluents include distilled water, physiological phosphate-buffered saline, Ringer's solutions, dextrose solution, and Hank's solution.

Pharmaceutical compositions can also include large, slowly metabolized macromolecules such as proteins, polysaccharides such as chitosan, polylactic acids, polyglycolic acids and copolymers (such as latex functionalized sepharose, agarose, cellulose), polymeric amino acids, amino acid copolymers, and lipid aggregates (such as oil droplets or liposomes).

The pharmaceutical composition of the present invention can further contain an adjuvant. One class of preferred adjuvants is aluminum salts, such as aluminum hydroxide, aluminum phosphate, or aluminum sulfate. Such adjuvants can be used with or without other specific immunostimulating agents such as MPL or 3-DMP, QS-21, flagellin, attenuated Salmonella (e.g., Salmonella typhimurium), polymeric or monomeric amino acids such as polyglutamic acid or polylysine, or pluronic polyols. Oil-in-water emulsion formulations are also suitable adjuvants that can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™, or other bacterial cell wall components). A suitable oil-in-water emulsion is MF59 (containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.) as described in WO 90/14837 to Van Nest et al., which is hereby incorporated by reference in its entirety. Other suitable oil-in-water emulsions include SAF (containing 10% Squalene, 0.4% Tween 80, 5% pluronic-blocked polymer L121, and thr-MDP, either microfluidized into a submicron emulsion or vortexed to generate a larger particle size emulsion) and Ribi™ adjuvant system (RAS; containing 2% squalene, 0.2% Tween 80, and one or more bacterial cell wall components). Another class of preferred adjuvants is saponin adjuvants, such as Stimulon™ (QS-21) or particles generated therefrom such as ISCOMs (immunostimulating complexes) and ISCOMATRIX. Other suitable adjuvants include incomplete or complete Freund's Adjuvant (IFA), cytokines, such as interleukins (IL-1, IL-2, and IL-12), macrophage colony stimulating factor (M-CSF), lysolecithin, tumor necrosis factor (TNF), and liposome polycation DNA particles. Such adjuvants are generally available from commercial sources.

In another embodiment of the present invention, the pharmaceutical composition further includes a delivery vehicle. Suitable delivery vehicles include, but are not limited to biodegradable microspheres, microparticles, nanoparticles, liposomes, collagen minipellets, and cochleates.

Another aspect of the present invention relates to a kit for treating psychosis in a subject. The kit includes a melatonergic agonist, a “typical” anti-psychotic drug, and instructions for treating psychosis in the subject by administering the melatonergic agonist and the “typical” anti-psychotic drug.

Melatonergic agonsists and “typical” anti-psychotic drugs are described above.

In one embodiment, the melatonergic agonist is in the form of a first pharmaceutical composition comprising the melatonergic agonist and a pharmaceutically acceptable carrier. Pharmaceutical compositions as well as pharmaceutically acceptable carriers are described above.

In a further embodiment, the “typical” anti-psychotic drug is in the form of a second pharmaceutical composition comprising the “typical” anti-psychotic drug and a pharmaceutically acceptable carrier.

A further aspect of the present invention relates to a method of treating a subject for schizophrenia. This method involves administering to the subject (i) a melatonergic agonist and (ii) a “typical” antipsychotic to treat the subject for schizophrenia.

As used herein, “treatment” or “treating” is an approach for obtaining a beneficial or desired result, including clinical results (e.g., reducing the severity or duration of, stabilizing the severity of, or eliminating one or more symptoms (biochemical, histological, and/or behavioral) of schizophrenia). For purposes of this invention, beneficial or desired results include, but are not limited to, alleviation of symptoms associated with schizophrenia, diminishment of the extent of the symptoms associated with schizophrenia, preventing a worsening of the symptoms associated with schizophrenia, including positive and/or negative and/or disorganized symptoms. Treatment embraces increasing the quality of life of those suffering from schizophrenia, decreasing the dose of other medications required to treat schizophrenia, delaying the progression of schizophrenia, and/or prolonging survival of schizophrenia patients.

In one embodiment, the subject has failed treatment with a “typical” antipsychotic.

Effective doses of the compositions of the present invention vary depending upon many different factors, including means of administration, target site, physiological state of the subject, and other medications administered. Usually, the subject is a mammal, including a human. Treatment dosages need to be titrated to optimize safety and efficacy and could involve oral treatment.

Compositions of the present invention can be administered by parenteral, topical, intravenous, oral, subcutaneous, intraperitoneal, intranasal, or intramuscular means for therapeutic treatment.

Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PYP). If desired, disintegrating agents can be added, such as a cross-linked polyvinyl pyrrolidone, agar, or alginic acid or a salt thereof such as sodium alginate. Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

The pharmaceutical agents of the present invention may be formulated for parenteral administration. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the pharmaceutical agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present invention can also be achieved using infusion pump devices such as those described by Medtronic, Northridge, Calif. Such devices allow continuous infusion of desired compounds avoiding multiple injections and multiple manipulations.

In addition to the formulations described previously, the compositions of the present invention may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

### EXAMPLES

The examples below are intended to exemplify the practice of the present invention but are by no means intended to limit the scope thereof.

### Example 1

### HistoReceptOmics Fingerprints for Drugs and Drug-like Compounds

Materials and Methods

Chemical Library

Chemical structures were obtained from four different sources: DrugBank v.2.5, National Cancer Institute Diversity Set II, PubChem, and ChEMBL 14. Overall, 1,141,434 unique nonoverlapping chemical structures were uploaded into the data warehouse.

Compound-Compound Associations

Compound-compound associations were evaluated as a chemical similarity measure between two compounds and derived as Tanimoto distance between their molecular fingerprints as implemented in ICM molecular modeling software (Grigoryan et al., “Spatial Chemical Distance Based on Atomic Property Fields,” J. Comput. Aided Mol. Des. 24(3): 173-182 (2010), which is hereby incorporated by reference in its entirety). Briefly, given a molecule, all linear and non-linear fragments of different size were enumerated and hashed into a bit string called a fingerprint. (NOTE: this kind of fingerprint is a discrete computer science entity, different from the fingerprints referred to elsewhere in this application for drug action, which are conceptual in nature) The Tanimoto coefficient, T, for two fingerprints was calculated as the number of bits in which they differ divided by the number of non-zero bits they have in common. The Tanimoto distance was defined as 1−T.

Protein Library

A set of 20,266 human protein targets were imported from the XML release of UniProt. Molecular targets known to be involved primarily in pharmacokinetics of drug action, such as the cytochromes, were removed from the list in order to concentrate the results on mechanism of action.

Compound-Target Associations

To connect calculated compound-compound associations to the protein targets, a set of experimentally obtained compound-target associations were imported into the data warehouse.

Source of In Vitro Binding Data

Over 300,000 instances of direct experimental evidence of compound-target binding with corresponding binding constants were derived from ChEMBL, with confidence score >7, relation noted as “=” or “<”. All compound-target associations obtained from ChEMBL can be easily tracked to their original scientific publications. In addition, compound-target binding can be determined using physical binding assays well known in the art.

Protein Target-Gene Expression Associations

Gene expression patterns of protein targets from a diverse set of tissues and cell types were derived from the “GeneAtlas U133A, gcrma” dataset (Su et al. “A Gene Atlas of the Mouse and Human Protein-encoding Transcriptomes,” Proc. Natl. Acad. Sci. USA 101(16): 6062-6067 (2004), which is hereby incorporated by reference in its entirety) via the BioGPS web-tool (Wu et al., “BioGPS: An Extensible and Customizable Portal for Querying and Organizing Gene Annotation Resources,” Genome Biol. 10(11): R130 (2009); Wu et al., “BioGPS and MyGene.info: Organizing Online, Gene-centric Information,” Nucleic Acids Res. 41: D561-5 (2012), each of which are hereby incorporated by reference in their entirety). Gene expression patterns of protein targets can also be obtained from any database that contains protein expression data, such as NCBI Gene Expression Omnibus (GEO). In addition, protein target expression data can be obtained by physical methods of protein analysis. When it is desirable to measure the expression level of a gene by measuring the level of protein expression, any protein binding or immunodetection based assay known in the art can be used. In a protein binding based assay, an antibody or other agent that selectively binds to a protein is used to detect the amount of that protein expressed in a sample. For example, the level of expression of a protein can be measured using methods that include, but are not limited to, western blot, immunoprecipitation, enzyme-linked immunosorbent assay (ELISA), radioimmunoassay (RIA), fluorescent activated cell sorting (FACS), immunohistochemistry, immunocytochemistry, or any combination thereof. Also, antibodies, aptamers, or other ligands that specifically bind to a protein can be affixed to so-called “protein chips” (protein microarrays) and used to measure the level of expression of a protein in a sample. Alternatively, assessing the level of protein expression can involve analyzing one or more proteins by two-dimensional gel electrophoresis, mass spectroscopy (MS), matrix-assisted laser desorption/ionization-time of flight-MS (MALDI-TOE), surface-enhanced laser desorption ionization-time of flight (SELDI-TOF), high performance liquid chromatography (HPLC), fast protein liquid chromatography (FPLC), multidimensional liquid chromatography (LC) followed by tandem mass spectrometry (MS/MS), protein chip expression analysis, gene chip expression analysis, and laser densitometry, or any combinations of these techniques. If, for a given gene, data from multiple probes were available, the median of those values was used. For each target protein, the level of expression in each tissue was normalized with regard to its level of expression in all tissues of the dataset and projected into the Z-score.

Tissue-Specific Drug-Target Scoring

Each drug-target association in each tissue is scored with regard to a bioactivity (affinity) of a given drug to a given protein target and an expression level of that same protein target in a given tissue (Z). The score is calculated as following:

Score=−log₁₀Affinity×Z

Data Access

The data is accessible via the world-wide web (drugable.com). A flexible free-text search index is available for common names of compounds and targets, medical conditions, etc. A chemical drawer allows the user to search by chemical similarity or substructure.

For example, when searching by compound common name or chemical structure, the user is presented with a compound chemical structure, compound information (number of hydrogen bond donors and hydrogen bond acceptors, number of rotatable bonds, number of rings, walden-crippen LogP, indication, pharmacology, mechanism of action etc.), and a table of compound-target associations available for this specific compound. The resulting table gives a list of protein targets that a compound of interest has reported or predicted affinity against, including protein target UniProt accession ID, the measured activity value and type, and a link to the original publication from which the value was obtained. Note that all the experimentally obtained activities are displayed in nM. In addition, a list of compounds that are chemically similar to the compound of interest is also presented. Furthermore, levels of expression for all genes corresponding to the protein targets that the compound of interest has reported or predicted affinity against in each tissue are presented as a hit map.

Alternatively, the user may want to search for a particular protein of interest. In this case, the user is presented with details of the protein target, such as X-ray structure (if available), protein name synonyms, gene names, organism this protein belongs to, and UniProt accession ID. Also, a table of compound-target associations available for this specific protein of interest is displayed. In addition, the levels of expression for a gene corresponding to a protein of interest in all tissues are presented.

Furthermore, the user may search for a medical condition of interest. In this case, the user is presented with a list of drugs/drug-like compounds as well as molecular (e.g., protein) targets associated with this medical condition.

Finally, all the results obtained by querying the data are summarized in an EXCEL® spreadsheet and can be easily downloaded into a local machine.

### Discussion

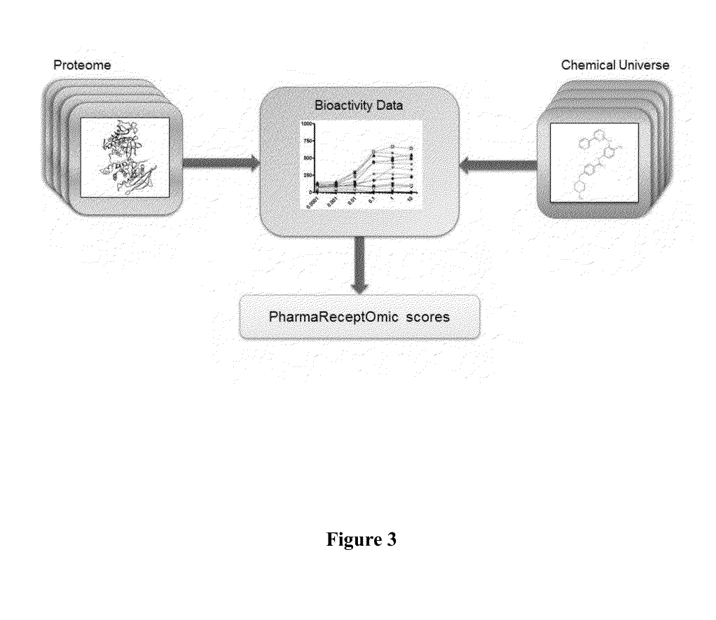

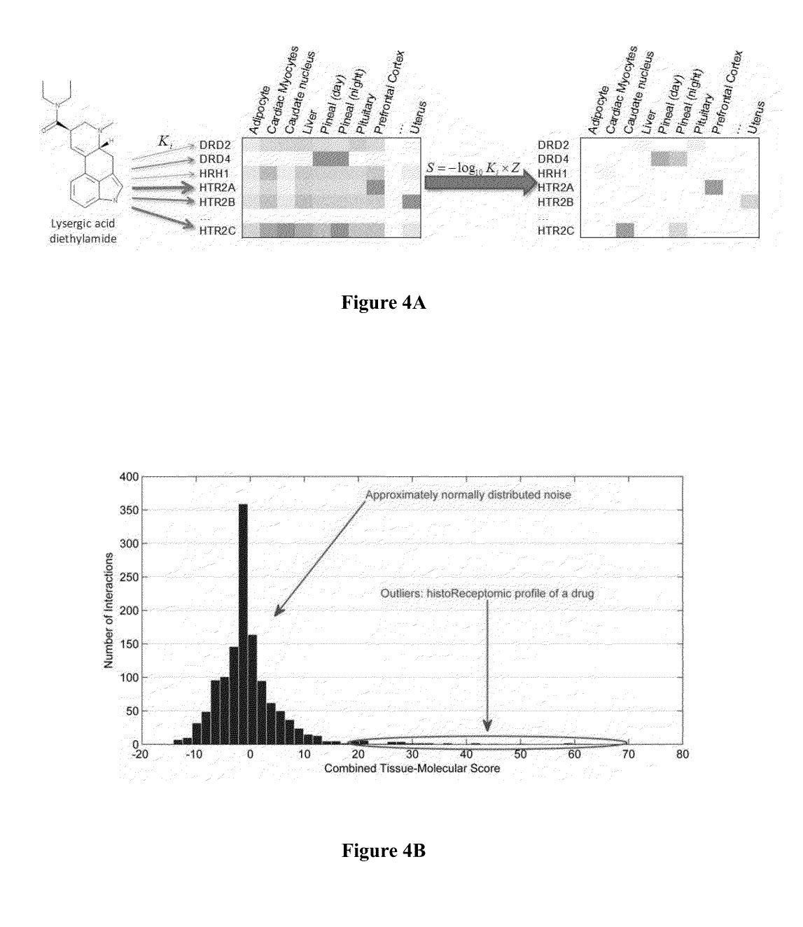

A two-step approach for the development of HistoReceptOmic fingerprints was developed. In the first step, bioactivity probability scores for each compound-target pair are calculated. This is done utilizing compound-target affinity obtained experimentally (FIG. 3). In the second step, a tissue-specific compound-target protein score is calculated using the bioactivity probability score and the tissue-specific gene expression score for each receptor in each tissue (FIG. 4A). For gene expression, microarray data characterizing the tissue-specific expression levels of protein targets derived from the BioGPS database was processed in a way that for each target protein, the level of expression in each tissue was normalized with regard to its level of expression in all tissues of the dataset and projected into the Z-score. Each drug-target association in each tissue is scored with regard to a bioactivity probability score of a given drug compound to a given molecular target and an expression level of that same molecular target in a given tissue (Z-score). The final HistoReceptOmics score is calculated as following:

Score(S)=−log₁₀Affinity(Kᵢ)×Z.

For any given drug, a lot of false positive HistoReceptOmics scores are generated, and it becomes necessary to identify the outlying true positives from this distribution in order to identify physiologically relevant drug compound—target interactions. The Generalized ESD outlier detection method (Rosner, “Percentage Points for a Generalized ESD Many-Outlier Procedure,” Technometrics 25(2): 165-172 (1983), which is hereby incorporated by reference in its entirety) was used to generate a p-value for the significance of each score (FIG. 4B). The set of all significant scores comprises the HistoReceptOmics fingerprint of the drug.

All the data used in this study were integrated into a unified web-searchable database at drugable.com. This database serves as a backbone for the growth of the HistoReceptOmics approach due to increasing bioactivity data and custom genetic backgrounds, other than BioGPS.

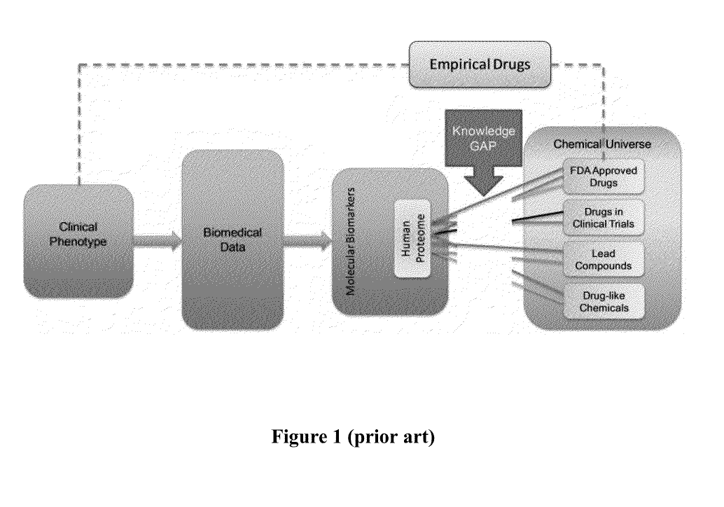

Since these HistoReceptOmics fingerprints contain both a specific pattern of targets and a specific pattern of tissues, they can be matched to complex biomarkers of disease derived from exhaustive molecular profiling. This novel approach thus potentially fills a currently existing gap between numerous ‘Omics’ data and drugs/drug-like compounds (FIG. 1). Furthermore, the integration of gene expression data provides a strategy for selecting only relevant interactions, since it does not matter whether a certain compound has high affinity for a certain receptor if this receptor is not expressed in a tissue/organ that is affected by a disease.

### Example 2

### Chemistry-Based Molecular Signature for CNS Sub-System Activity Underlying the Atypia of Clozapine

The vast majority of drugs that are used to treat psychiatric disorders were discovered by serendipity. The main reason is that psychiatric diseases are not easily studied organically at the levels of molecular, cellular, and, particularly, animal model biology. Psychiatry has a diagnostic and classification system that is, in general, not based on etiology, pathophysiology, epidemiology, or genetics, but rather on a constellation of human behavioral signs and symptoms (Gould et al., “Psychiatric Endophenotypes and the Development of Valid Animal Models,” Genes Brain Behav 5:113-119 (2006); Agid et al., “How Can Drug Discovery for Psychiatric Disorders be Improved?” Nat. Rev. Drug Discov. 6:189-201 (2007), each of which is hereby incorporated by reference in its entirety). Thus, understanding of the bio-mechanistic aspects of these disorders remains very limited despite many years of research, and, concomitantly, the record of drug development in psychiatry has been historically quite poor.

Specific chemical entities are, however, used successfully to treat distinct psychiatric conditions, and certain chemical entities exhibit consistently superior therapeutic benefits over others used to treat the same condition. Thus, there are defined molecular entities—psychiatric drugs—to which phenotypes in psychiatry may be matched. Moreover, the drugs themselves are an unexplored avenue available to define the organic basis, or molecular signatures, of psychiatric phenotypes. A good example of a precise psychiatric phenotype is illustrated by the therapeutic use of the anti-psychotic drug clozapine (CLOZARIL™).

Anti-psychotics have historically been grouped observationally according to both their pattern of clinical action and their suspected (but not proven) mechanism of action. The original antipsychotic drugs such as chlorpromazine (THORAZINE™) and haloperidol are considered “typical,” exhibiting reliable antipsychotic actions accompanied by consistent extrapyramidal and endocrine side effects that have been ascribed to their high affinity dopamine D2 receptor antagonism (Nord et al., “Antipsychotic Occupancy of Dopamine Receptors in Schizophrenia,” CNS Neuroscience & Therapeutics 17:97-103 (2011), which is hereby incorporated by reference in its entirety). Second generation “atypical” anti-psychotics are considered to have lower affinity for D2 receptors and greater affinities for other receptors such as serotonin and norepinephrine (Miyamoto et al., “Treatments for Schizophrenia: A Critical Review of Pharmacology and Mechanisms of Action of Antipsychotic Drugs,” Molecular Psychiatry 10:79-104 (2005), which is hereby incorporated by reference in its entirety). Clozapine is an “atypical” neuroleptic that is often effective in patients that have failed typical anti-psychotics (suggesting a partly different mechanism of action), and it is associated with fewer extrapyramidal and possibly fewer cognitive side effects (Meltzer, H. Y., “Treatment-resistant Schizophrenia—The Role of Clozapine,” Curr. Med. Res. Opin. 14:1-20 (1997), which is hereby incorporated by reference in its entirety). Clozapine's “atypia,” originally ascribed by consensus to its high-affinity 5-HT2A antagonism (Kapur et al., “Serotonin-dopamine Interaction and its Relevance to Schizophrenia,” The American Journal of Psychiatry 153:466-476 (1996), which is hereby incorporated by reference in its entirety), is likely poly-pharmacologic, though its precise basis remains unknown. Although it appears to be a superior drug for psychosis, clozapine is not first-line therapy because it idiosyncratically causes agranulocytosis, which can be fatal without supportive medical care (Wu et al., “BioGPS: An Extensible and Customizable Portal for Querying and Organizing Gene Annotation Resources,” Genome Biol. 10:R130 (2009), which is hereby incorporated by reference in its entirety), and has other significant side effects, including orthostatic hypotension and hypersalivation. Although anti-psychotic drugs are associated with various individual receptors in the literature, these associations have not sufficiently illuminated their mechanism of action, and a polypharmacologic view of these drugs is probably more informative.

It was hypothesized that a novel combined CNS subsystem-molecular signature for the mechanism of action of the atypia of clozapine by subtracting chlorpromazine's drug-action profile could be inferred from clozapine's drug action profile. A key hypothesis tested was that understanding drug action via tissue-target profiles is more productive than tissue-agnostic methods for highly complex phenotypes. Notably, the input data for the approach was solely the bioactivities of drugs against the entire collection of human receptors combined with the gene expression levels of those receptors in variety of human tissues. Thus, the results correlate the molecular findings directly to human psychiatric phenotypes. As such, this approach may yield previously obscure organic bases for psychiatric phenotypes that no other investigative method can obtain.

A conceptual framework to integrate drug-target affinity data with the expression data of the target in specific tissues was designed, as described supra in Example 1. This framework relates the polypharmacologic profile of a drug and the expression level of the entire set of the receptors in every human tissue. The combined tissue-target scoring of drugs was performed as described supra. Using this approach, physiologically relevant bioactivities are identified by those tissue specific drug-target interactions that have high combined scores.

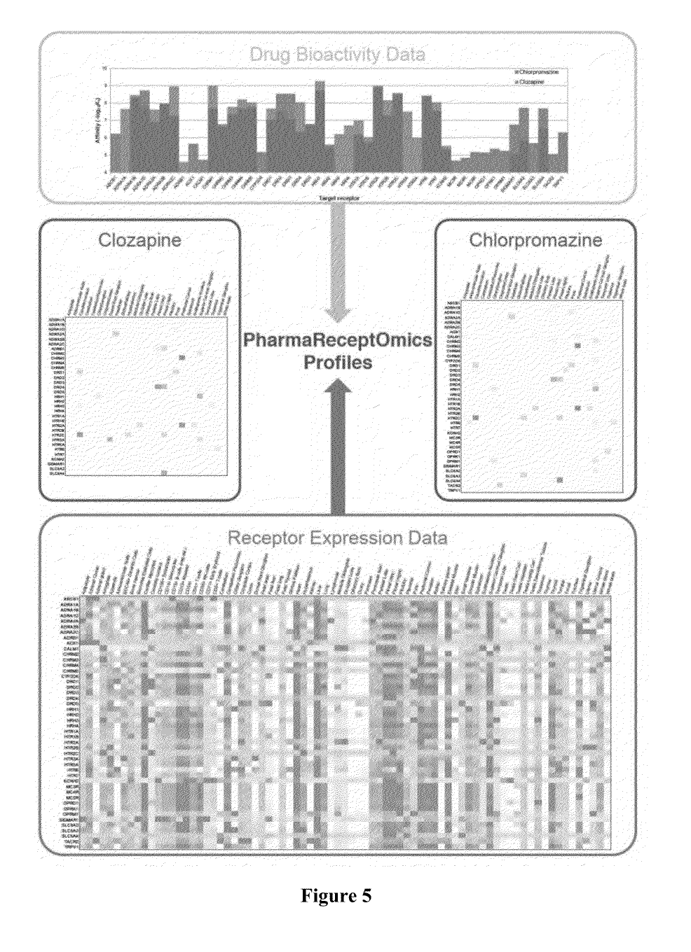

The entire dataset of potential human receptors of clozapine and chlorpromazine was analyzed, and reliable affinity (Ki) data for 34 proteins targeted by clozapine and 41 proteins targeted by chlorpromazine was obtained (FIG. 5 and Table 1).

TABLE 1

| | Ki | | | |
|---|---|---|---|---|
| Drug | (nM) | GeneSymbol | Name | Organism |
| CLOZAPINE | 22 | Adra1a | Alpha-1a | Rattus norvegicus |
| | | | adrenergic receptor | |
| CLOZAPINE | 3.59 | Adra1b | Alpha-1b | Rattus norvegicus |
| | | | adrenergic receptor | |
| CLOZAPINE | 17 | ADRA1D | Alpha-1d | Homo sapiens |
| | | | adrenergic receptor | |
| CLOZAPINE | 24 | ADRA2A | Alpha-2a | Homo sapiens |
| | | | adrenergic receptor | |
| CLOZAPINE | 11 | ADRA2B | Alpha-2b | Homo sapiens |
| | | | adrenergic receptor | |
| CLOZAPINE | 1.14 | ADRA2C | Alpha-2c | Homo sapiens |
| | | | adrenergic receptor | |
| CLOZAPINE | 26000 | Adrb1 | Beta-1 adrenergic | Rattus norvegicus |
| | | | receptor | |
| CLOZAPINE | 0.98 | CHRM1 | Muscarinic | Homo sapiens |
| | | | acetylcholine | |
| | | | receptor M1 | |
| CLOZAPINE | 169 | CHRM2 | Muscarinic | Homo sapiens |
| | | | acetylcholine | |
| | | | receptor M2 | |
| CLOZAPINE | 17 | CHRM3 | Muscarinic | Homo sapiens |
| | | | acetylcholine | |
| | | | receptor M3 | |
| CLOZAPINE | 6.3 | CHRM4 | Muscarinic | Homo sapiens |
| | | | acetylcholine | |
| | | | receptor M4 | |
| CLOZAPINE | 9.53 | CHRM5 | Muscarinic | Homo sapiens |
| | | | acetylcholine | |
| | | | receptor M5 | |
| CLOZAPINE | 21.38 | DRD1 | Dopamine D1 | Homo sapiens |
| | | | receptor | |
| CLOZAPINE | 28 | DRD2 | Dopamine D2 | Homo sapiens |
| | | | receptor | |
| CLOZAPINE | 88 | DRD3 | Dopamine D3 | Homo sapiens |
| | | | receptor | |
| CLOZAPINE | 9 | DRD4 | Dopamine D4 | Homo sapiens |
| | | | receptor | |
| CLOZAPINE | 198 | DRD5 | Dopamine D5 | Homo sapiens |
| | | | receptor | |
| CLOZAPINE | 0.571 | HRH1 | Histamine H1 | Homo sapiens |
| | | | receptor | |
| CLOZAPINE | 3550 | HRH2 | Histamine H2 | Homo sapiens |
| | | | receptor | |
| CLOZAPINE | 631 | HRH3 | Histamine H3 | Homo sapiens |
| | | | receptor | |
| CLOZAPINE | 199.53 | HRH4 | Histamine H4 | Homo sapiens |
| | | | receptor | |
| CLOZAPINE | 101 | HTR1A | Serotonin 1a (5- | Homo sapiens |
| | | | HT1a) receptor | |
| CLOZAPINE | 959 | Htr1b | Serotonin 1b (5- | Rattus norvegicus |
| | | | HT1b) receptor | |
| CLOZAPINE | 1.2 | HTR2A | Serotonin 2a (5- | Homo sapiens |
| | | | HT2a) receptor | |
| CLOZAPINE | 7.18 | HTR2B | Serotonin 2b (5- | Homo sapiens |
| | | | HT2b) receptor | |
| CLOZAPINE | 3.16 | HTR2C | Serotonin 2c (5- | Homo sapiens |
| | | | HT2c) receptor | |
| CLOZAPINE | 32 | Htr3a | Serotonin 3a (5- | Mus musculus |
| | | | HT3a) receptor | |
| CLOZAPINE | 1000 | HTR5A | Serotonin 5a (5- | Homo sapiens |
| | | | HT5a) receptor | |
| CLOZAPINE | 4 | HTR6 | Serotonin 6 (5- | Homo sapiens |
| | | | HT6) receptor | |
| CLOZAPINE | 9 | HTR7 | Serotonin 7 (5- | Homo sapiens |
| | | | HT7) receptor | |
| CLOZAPINE | 3080 | KCNH2 | HERG | Homo sapiens |
| CLOZAPINE | 8500 | SIGMAR1 | Sigma opioid | Homo sapiens |
| | | | receptor | |
| CLOZAPINE | 1458 | SLC6A2 | Norepinephrine | Homo sapiens |
| | | | transporter | |
| CLOZAPINE | 290 | SLC6A4 | Serotonin | Homo sapiens |
| | | | transporter | |
| CHLORPROMAZINE | 600 | ABCB1 | P-glycoprotein 1 | Homo sapiens |
| CHLORPROMAZINE | 4.96 | Adra1b | Alpha-1b | Rattus norvegicus |
| | | | adrenergic receptor | |
| CHLORPROMAZINE | 1.96 | ADRA1D | Alpha-1d | Homo sapiens |
| | | | adrenergic receptor | |
| CHLORPROMAZINE | 132 | ADRA2A | Alpha-2a | Homo sapiens |
| | | | adrenergic receptor | |
| CHLORPROMAZINE | 12 | ADRA2B | Alpha-2b | Homo sapiens |
| | | | adrenergic receptor | |
| CHLORPROMAZINE | 54 | ADRA2C | Alpha-2c | Homo sapiens |
| | | | adrenergic receptor | |
| CHLORPROMAZINE | 2300 | AOX1 | Aldehyde oxidase | Homo sapiens |
| CHLORPROMAZINE | 19280 | CALM1 | Calmodulin | Homo sapiens |
| CHLORPROMAZINE | 20 | CHRM1 | Muscarinic | Homo sapiens |
| | | | acetylcholine | |
| | | | receptor M1 | |
| CHLORPROMAZINE | 232 | CHRM2 | Muscarinic | Homo sapiens |
| | | | acetylcholine | |
| | | | receptor M2 | |
| CHLORPROMAZINE | 44 | CHRM3 | Muscarinic | Homo sapiens |
| | | | acetylcholine | |
| | | | receptor M3 | |
| CHLORPROMAZINE | 21 | CHRM4 | Muscarinic | Homo sapiens |
| | | | acetylcholine | |
| | | | receptor M4 | |
| CHLORPROMAZINE | 18 | CHRM5 | Muscarinic | Homo sapiens |
| | | | acetylcholine | |
| | | | receptor M5 | |
| CHLORPROMAZINE | 7000 | CYP2D6 | Cytochrome P450 | Homo sapiens |
| | | | 2D6 | |
| CHLORPROMAZINE | 96 | DRD1 | Dopamine D1 | Homo sapiens |
| | | | receptor | |
| CHLORPROMAZINE | 3 | DRD2 | Dopamine D2 | Homo sapiens |
| HYDROCHLORIDE | | | receptor | |
| CHLORPROMAZINE | 3 | DRD3 | Dopamine D3 | Homo sapiens |
| | | | receptor | |
| CHLORPROMAZINE | 479 | DRD4 | Dopamine D4 | Homo sapiens |
| | | | receptor | |
| CHLORPROMAZINE | 172 | DRD5 | Dopamine D5 | Homo sapiens |
| | | | receptor | |
| CHLORPROMAZINE | 1.96 | HRH1 | Histamine H1 | Homo sapiens |
| | | | receptor | |
| CHLORPROMAZINE | 2582 | HRH2 | Histamine H2 | Homo sapiens |
| | | | receptor | |
| CHLORPROMAZINE | 673 | HTR1A | Serotonin 1 (5- | Homo sapiens |
| | | | HT1a) receptor | |
| CHLORPROMAZINE | 1837 | Htr1b | Serotonin 1 (5- | Rattus norvegicus |
| | | | HT1b) receptor | |
| CHLORPROMAZINE | 1.1 | HTR2A | Serotonin 2a (5- | Homo sapiens |
| | | | HT2a) receptor | |
| CHLORPROMAZINE | 52 | HTR2B | Serotonin 2b (5- | Homo sapiens |
| | | | HT2b) receptor | |
| CHLORPROMAZINE | 2.74 | HTR2C | Serotonin 2c (5- | Homo sapiens |
| | | | HT2c) receptor | |
| CHLORPROMAZINE | 4 | HTR6 | Serotonin 6 (5- | Homo sapiens |
| | | | HT6) receptor | |
| CHLORPROMAZINE | 27 | HTR7 | Serotonin 7 (5- | Homo sapiens |
| | | | HT7) receptor | |
| CHLORPROMAZINE | 4774.3 | KCNH2 | HERG | Homo sapiens |
| CHLORPROMAZINE | 21244 | MC3R | Melanocortin | Homo sapiens |
| | | | receptor 3 | |
| CHLORPROMAZINE | 14619 | MC4R | Melanocortin | Homo sapiens |
| | | | receptor 4 | |
| CHLORPROMAZINE | 7145 | MC5R | Melanocortin | Homo sapiens |
| | | | receptor 5 | |
| CHLORPROMAZINE | 7365 | OPRD1 | Delta opioid | Homo sapiens |
| | | | receptor | |
| CHLORPROMAZINE | 4433 | OPRK1 | Kappa opioid | Homo sapiens |
| | | | receptor | |
| CHLORPROMAZINE | 5844 | OPRM1 | Mu opioid receptor | Homo sapiens |
| CHLORPROMAZINE | 189 | SIGMAR1 | Sigma opioid | Homo sapiens |
| | | | receptor | |
| CHLORPROMAZINE | 19 | SLC6A2 | Norepinephrine | Homo sapiens |
| | | | transporter | |
| CHLORPROMAZINE | 2100 | SLC6A3 | Dopamine | Homo sapiens |
| | | | transporter | |
| CHLORPROMAZINE | 21 | SLC6A4 | Serotonin | Homo sapiens |
| | | | transporter | |
| CHLORPROMAZINE | 9065 | TACR2 | Neurokinin 2 | Homo sapiens |
| | | | receptor | |
| CHLORPROMAZINE | 520 | Trpv1 | Vanilloid receptor | Rattus norvegicus |

These affinity data were then combined with gene expression data for each protein target in 77 normal human tissues to obtain HistoReceptOmics signatures for both drugs (FIG. 5).

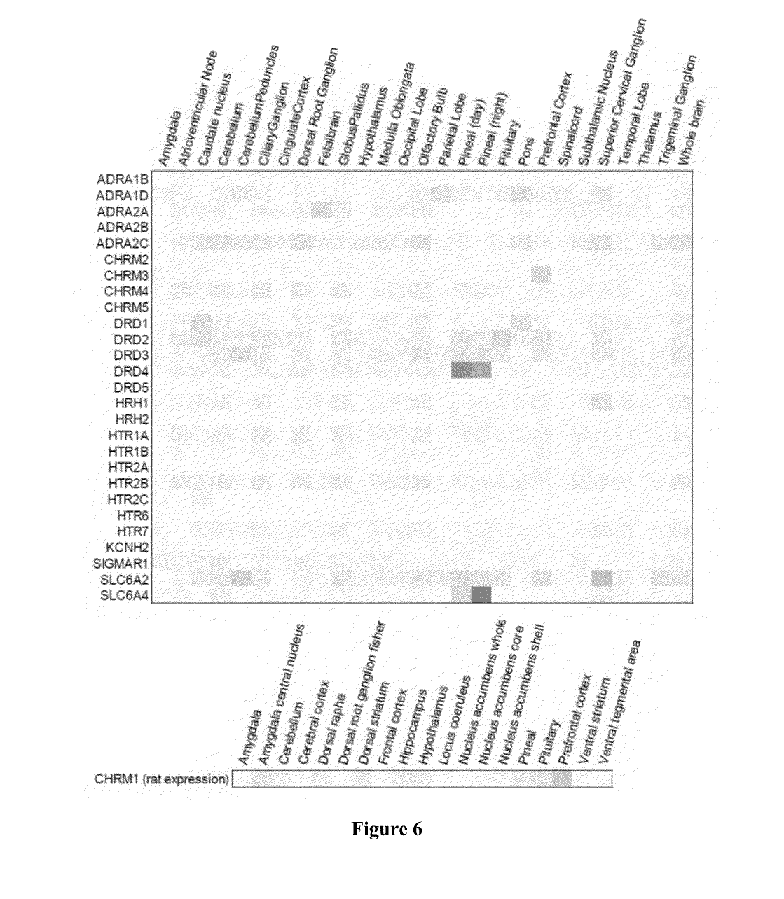

The common anti-psychotic effect of clozapine and chlorpromazine is represented by the overlap between the two signatures, which was determined to be serotonin 2a (5-HT2a) and 2c (5-HT2c) receptors in the prefrontal cortex and the caudate nucleus, respectively (FIG. 6). This defines a subsystem target signature for the common anti-psychotic effect of the two drugs. The notable targets specific to clozapine are dopamine D4 receptor in pineal gland, the muscarinic acetylcholine receptor M1 in the prefrontal cortex, the histamine H1 receptor in superior cervical ganglion, and the muscarinic acetylcholine receptor M3 (CHRM3) in the prefrontal cortex (FIG. 6). On the other hand, serotonin transporter SLC6A4 in the night phase of the pineal gland was the only receptor exclusive to chlorpromazine Although the aforementioned receptors interact with both drugs with at least a micromolar affinity, the affinity of one drug to any given receptor is at least two fold higher than to the other (FIG. 6).

Prior polypharmacologic approaches to drug action have successfully predicted new targets for known psychiatric drugs and side-effects of drugs (Lounkine et al., “Large-scale Prediction and Testing of Drug Activity on Side-effect Targets,” Nature 486:361-367 (2012); Keiser et al. “Predicting New Molecular Targets for Known Drugs,” Nature 462:175-181 (2009), each of which is hereby incorporated by reference in its entirety). These studies demonstrate that understanding of drug action based on the full set of relevant targets is superior to the single-target view. However, drug action is incontrovertibly the product of both chemical activity on targets and the expression pattern of those targets in specific tissues in the human body. This combined tissue-target view of drug action has not previously been investigated on a large scale. Here, the tissue-target approach was applied to derive novel signatures for the functional biological (brain) system underlying the atypia of clozapine. The signature departs from the consensus receptor-target based view of the action of anti-psychotics and clozapine's atypia in several important ways. First, the serotonin 5-HT2a receptor is classified by the approach as a component of the common anti-psychotic effect of clozapine and chlorpromazine, and is not observed to be specific to clozapine. Notably, 5-HT2a is the target receptor of lysergic acid diethylamide (LSD), which produces symptoms in normal individuals similar to the psychosis symptoms in schizophrenia (Osmond et al., “Schizophrenia: A New Approach,” J. Ment. Sci. 98: 309-315 (1952), which is hereby incorporated by reference in its entirety). Interestingly, D2 receptors, which are widely cited as being involved in both psychosis and the action of these drugs, do not stand out as the strongest contributors to the action of either drug, although they appear on the list at more sensitive p-values.

Furthermore, the specific sites of action of the receptors are part of the subsystem signature that was derived, and, in the case of 5-HT2a, it was found that this specific site is the prefrontal cortex (“PFC”). The PFC has long been implicated in schizophrenia: the only data deriving from direct observation of the disease in humans, neuroimaging, localizes schizophrenia specific brain activity to the PFC (Lawrie et al., “Neuroimaging and Molecular Genetics of Schizophrenia: Pathophysiological Advances and Therapeutic Potential,” Br. J. Pharmacol. 153 Suppl 1: S120-4 (2008), which is hereby incorporated by reference in its entirety).

Another major departure from consensus views is that the activity of the serotonin 5-HT2c receptor in the caudate is also associated with the bioactivity of both drugs. This association has not previously been accepted as a primary component of psychosis. The interaction with the caudate, a component of the basal ganglia, is interesting because the caudate is both involved in the pathogenesis of schizophrenia and associates with motor side effects, which fits with evidence on the drugs (Chakos et al., “Caudate Nuclei Volumes in Schizophrenic Patients Treated With Typical Antipsychotics or Clozapine.” Lancet 345:456-457 (1995), which is hereby incorporated by reference in its entirety).

The signature for clozapine's atypia strongly implicates D4 receptors in the pineal gland, which produces the hormone melatonin and thus strongly influences mood via circadian rhythms. The basis for clozapine's effects on mood were previously unknown. Indeed, melatonin has previously been studied for its mood stabilizing effects and the first melatonergic drug for the treatment of depression has been approved for human use (de Bodinat et al., “Agomelatine, the First Melatonergic Antidepressant: Discovery, Characterization and Development,” Nat. Rev. Drug Discov. 9:628-642 (2010), which is hereby incorporated by reference in its entirety). This suggests a new melatonergic hypothesis: Namely, that the combination of typical antipsychotics with melatonergic agonists may capture some of the beneficial atypical anti-psychosis effects of clozapine, while avoiding its limiting side effects such as agranulocytosis and orthostatic hypertension

Finally, the signature derived for the atypia of clozapine suggests that the bioactivities responsible for suppressing psychosis that is resistant to treatment with chlorpromazine are associated with clozapine's action on CHRM1 and CHRM2 in the prefrontal cortex. These receptors have not previously been singled out as targets for anti-psychotic treatment.

The signature that was derived provides some insight into a side effect of clozapine. Clozapine's atypical effect mapped to histamine receptor H1 in the SCG. This could explain its propensity to cause severe orthostatic hypotension, which is one reason clozapine must be started at a very low initial dose. The action in the SCG could also relate to hypersalivation—a debilitating side-effect of clozapine, since superior sympathetic ganglia and the hypothalamus control activity of the salivary nucleus in the brainstem. Interestingly, the most recent review of the evidence for hypersalivation treatment found that diphenhydramine, an antihistamine, was among the most effective treatments for clozapine-induced hypersalivation (Syed et al., “Pharmacological Interventions for Clozapine-Induced Hypersalivation,” Cochrane Database Syst. Rev. 16(3): CD005579 (2008), which is hereby incorporated by reference in its entirety).

As a prototype, this new approach of deriving a biological signature for a psychiatric phenotype could be used to investigate many phenotypes, but has several limitations. On the other hand, these current limitations provide paths for relevant perspectives and future improvements based on this first-of-class reported concept for data integration of combined receptor activity—receptor tissue expression protocols. First, the recorded bioactivities do not cover the space of all pairwise scores between these drugs and all proteins in the human proteome. This suggests that important targets could have been missed. Second, the gene expression patterns used were for normal tissues. It is known that gene expression in some of these tissues differs in afflicted individuals, due to the disease as well as its treatment. A new anatomic gene expression map based on schizophrenic individuals may improve the signature. Notably, the new brain mapping effort may provide a gene expression pattern for each of the 86 billion neurons in the human brain, allowing localization of the signature to specific neurons and circuits. Finally, all protein targets associated with drug metabolism and transport were eliminated, such as the cytochromes, in order to isolate signaling receptors and achieve a purely pharmacodynamic (mechanistic) result. However, some of the targets eliminated may have pharmacodynamic effects. Nevertheless, the results with the prototype has generated previously unsuspected clinico-pharmacologic hypotheses and suggests that chemistry-based biological signatures are a promising approach to exploring mechanism and developing biomarkers in complex diseases with obscure molecular bases such as mental health disorders.

The results also have general implications for informatics research. Molecular signatures of complex mechanisms of action are often derived from comparative genetic or proteomic profiling, but signatures derived in this way suffer from tissue agnosticism and the inability to distinguish a causal signature from a predictive/correlative signature (Nilsson et al., “On Reliable Discovery of Molecular Signatures,” BMC Bioinformatics 10:38 (2009); Statnikov et al., “Analysis and Computational Dissection of Molecular Signature Multiplicity,” PLoS Computational Biology 6:e1000790 (2010), each of which is hereby incorporated by reference in its entirety). Here, a signature from the drug used to treat the disease has been derived. As this signature is derived starting from the drug pharmacologically linked to the phenotype, it is a priori at least partially a causal molecular signature of the phenotype. The results are useful in pharmacoinformatics as a gold standard for comparison to causal signatures derived via network boundaries or other informatics means that attempt to derive these signatures within single domains such as gene expression or proteomics. Conversely, testing a genetic or proteome derived molecular signature with matched drugs might be argued as a novel method to establish causality for the signature.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method for characterizing the molecular basis of a drug compound's activity, said method comprising:
selecting a drug compound;
identifying with a molecular target computing device molecular targets with which the drug compound interacts, wherein said identifying comprises obtaining from a database comprising compound-target bioactivities a first and second molecular target for the drug compound; and
comparing the first and second molecular targets to expression levels of the first and second molecular targets to identify the molecular basis of the drug compound's activity.

2. The method according to claim 1, wherein the first and second molecular targets are proteins.

3. The method according to claim 2, wherein the proteins are selected from membrane proteins and soluble proteins.

4. The method according to claim 1, wherein said expression levels are in normal, non-disease tissue.

5. The method according to claim 1, wherein said expression levels are in disease tissue.

6. The method according to claim 1, wherein the drug compound has a known molecular target.

7. The method according to claim 1, wherein the drug compound does not have a known molecular target.

8. The method according to claim 1, wherein the drug compound is used to treat psychosis.

9. The method according to claim 1, wherein the compound-target bioactivities are associated with data ranking the relative strength and/or weakness of a molecular target's interaction with the drug compound to that of another molecular target's interaction with the drug compound.

10. The method according to claim 1, wherein said comparing is carried out using an algorithm.

11. The method according to claim 1 further comprising:
identifying a tissue in which the first and second molecular target exist.

12. The method according to claim 1 further comprising:
comparing the molecular basis of a first drug compound to the molecular basis of a second drug compound.

13. The method according to claim 12, wherein the second drug compound has known side effects not associated with the first drug compound.

14. A pharmaceutical composition comprising:
a melatonergic agonist;
a “typical” anti-psychotic drug; and
a pharmaceutically acceptable carrier.

15. The pharmaceutical composition according to claim 14, wherein said melatonergic agonist is selected from the group consisting of: agomelatine and ramelteon.

16. A kit for treating psychosis in a subject, said kit comprising:
a melatonergic agonist;
a “typical” anti-psychotic drug; and
instructions for treating psychosis in the subject by administering the melatonergic agonist and the “typical” anti-psychotic drug.

17. The kit according to claim 16, wherein the melatonergic agonist is in the form of a first pharmaceutical composition comprising the melatonergic agonist and a pharmaceutically acceptable carrier.

18. The kit according to claim 16, wherein the “typical” anti-psychotic drug is in the form of a second pharmaceutical composition comprising the “typical” anti-psychotic drug and a pharmaceutically acceptable carrier.

19. The kit according to claim 16, wherein said melatonergic agonist is selected from the group consisting of: agomelatine and ramelteon

20. A method of treating a subject for schizophrenia, said method comprising:
administering to the subject (i) a melatonergic agonist and (ii) a “typical” antipsychotic to treat the subject for schizophrenia.

21. The method according to claim 20, wherein said melatonergic agonist is selected from the group consisting of: agomelatine and ramelteon.

22. The method according to claim 20, wherein the subject has failed treatment with a “typical” antipsychotic.
